(19) United States
(12) Patent Application Publication
(10) Pub. No.: US 2010/0137346 A1
(43) Pub. Date: Jun. 3, 2010

(54) DESFERRITHIOCIN ANALOGUE ACTINIDE DECORPORATION AGENTS

(75) Inventor: Raymond J. Bergeron, JR., Gainsville, FL (US)

Correspondence Address: WOLF GREENFIELD & SACKS, P.C., 600 ATLANTIC AVENUE, BOSTON, MA, 02210-2206, US

(73) Assignee: University of Florida Research Foundation, Inc., Gainesville, FL (US)

(21) Appl. No.: 12/448,237

(22) PCT Filed: Dec. 12, 2007
(86) PCT No.: PCT/US2007/025377

§ 371 Date: Jan. 6, 2010

Related U.S. Application Data
(60) Provisional application No. 60/874,256, filed on Dec. 12, 2006.

(51) Int. Cl. A61K 31/435; A61K 31/195; A61K 31/426; A61P 39/04

(52) U.S. Cl. 514/277; 514/566; 514/365

(57) ABSTRACT

A pharmaceutical composition comprising a non-toxic effective amount of an actinide decorporation agent and a pharmaceutically acceptable carrier therefore, the actinide decorporation agent comprising a hexacoordinate desferrithiocin analogue capable of chelating an actinide in vivo and a method for removing an actinide from the tissue of a human or nonhuman mammal.

### CROSS REFERENCE TO RELATED APPLICATION

The subject matter of the subject invention is related to provisional application Ser. Nos. 60/874,256 filed on Dec. 12, 2006, and 60/966,539, filed on Mar. 15, 2007, the subject matter of which is incorporated herein in its entirety. Priority is claimed therefrom.

### GOVERNMENT SUPPORT CLAUSE

The invention was supported, in whole or in part, by grant No. DK49108 from the National Diabetes and Digestive and Kidney Diseases Advisory Council (NIDDK) of the National Institute of Health (NIH). The Government has certain rights in the invention. The entire contents and disclosures of each patent and reference disclosed herein are incorporated by reference.

### BACKGROUND OF THE INVENTION

There are any number of scenarios in which radioactive materials represent a credible threat in a terrorist attack, ranging from the so-called “dirty bomb” or RDD (radiological dispersion device), destruction of a nuclear reactor, to the unthinkable detonation of a thermal nuclear device. The management of these and other scenarios has been carefully explored by the Department of Homeland Security and the military. In most instances other than a thermal nuclear detonation, the issue reduces to a nuclear decontamination problem. While external contamination is easily managed, ingestion, inhalation or contamination of wounds with radionuclides becomes problematic. The current solution depends nearly entirely on the treatment of patients with chelators that sequester and permit the excretion of likely radioactive metals and/or administration of potassium iodide to prevent the uptake of radioactive iodide by the thyroid gland. While the list of potential metals is rather substantial, including but not limited to Am, Cf, Ce, Cs, Cu, Pu, Po, Sr, and U, it is not matched by a credible list of therapeutic chelators. Probably the most widely accepted chelator diethylenetriaminepentaacetic acid (DTPA) requires very prompt treatment with subcutaneous administration and presents with a number of side effects.

While subcutaneously administered chelators have a place, the lack of orally active ligands is of genuine concern in the case of a mass exposure. It would be difficult to manage large numbers of patients requiring protracted subcutaneous or intravenous administration of DTPA. In a definitive review by Raymond et al[Chem Rev 2003; 103:4207-4282] on the “Rational Design of Sequestering Agents for Plutonium and Other Actinides,” the authors underscore early on the similarities between Fe(III), Pu(IV), Am(III) and Eu(III), e.g., charge-to-radius ratio, biological transport, and distribution. In fact, although there are coordination differences between iron and the actinides, the similarities have served as drivers for the design of actinide ligands [Fukuda S. Chelating agents used for plutonium and uranium removal in radiation emergency medicine, Curr Med Chem 2005; 12:2765-2770; Paquet et al, Efficacy of 3,4,3-1i(1,2-HOPO) for decorporation of Pu, Am and U from rats injected intramuscularly with high-fired particles of MOX. Radiat Prot Dosimetry 2003; 105:521-525; Guilmette et al, Competitive binding of Pu and Am with bone mineral and novel chelating agents, Radiat Prot Dosimetry 2003; 105:527-534; Stradling et al, Recent developments in the decorporation of plutonium, americium and thorium, Radiat Prot Dosimetry 1998; 79:445-448; Santos et al, A cyclohexane-1,2-diyldinitrilotetraacetate tetrahydroxamate derivative for actinide complexation: Synthesis and complexation studies. J Chem Soc Dalton Trans 2000:4398-4402; Miller et al, Efficacy of orally administered amphipathic polyaminocarboxylic acid chelators for the removal of plutonium and americium: Comparison with injected Zn-DTPA in the rat, Radiat Prot Dosimetry 2005. E-published ahead of print.

Raymond et al, supra, overviews an impressive, systematic approach to the design of actinide chelators, leaving no doubt that while Fe(III) prefers to form hexacoordinate octahedral complexes, the actinides prefer octacoordinate dodecahedral complexes. However, it is also clear that his hydroxypyridinone (HOPO) hexacoordinate chelators will sequester and remove actinides from animals quite nicely. In fact, depending on the family of ligands, there can be small differences in efficiency between hexacoordinates and octacoordinates. In the same review, Raymond et al points out that a number of octacoordinate, hexacoordinate, and tetracoordinate catechol and HOPO ligands bind U(VI) [Durbin et al, Chelating agents for uranium(VI): 2. Efficacy and toxicity of tetradentate catecholate and hydroxypyridinonate ligands in mice, Health Phys 2000; 78:51 1-521.

It is an object of the invention to provide novel actinide decorporation agents.

### SUMMARY OF THE INVENTION

The above and other objects are realized by the present invention, one embodiment of which relates to the provision of desferrithiocin based chelating agents for decorporation of radionuclides.

More particularly, an embodiment of the invention relates to certain hexacoordinate desferrithiocin analogues, active as actinide decorporation agents and compositions and methods for removing actinides from human and non-human mammals.

### BRIEF DESCRIPTION OF THE DRAWINGS

FIGS. 1-4 set forth various chemical and physical characteristics and properties of the actinide decorporation agents of the invention.

### DETAILED DESCRIPTION OF THE INVENTION

Actinide decorporation agents utilized in the practice of the invention include any of Compounds 4-12, 17-21 (described hereinbelow) or compounds having the formula:

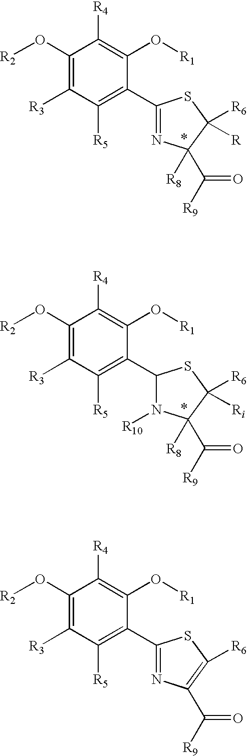

wherein:
R₁ is —H or an acyl group;
R₂ is —[(CH₂)ₙ—O]ₓ—[(CH₂)ₙ—O]_{y}—R′;
R₃, R₄ and R₅ are each independently —H, an alkyl group, or —OR₁₁;
R₆, R₇, and R₈ are each independently —H or an alkyl group;
R₉ is —OR₁₂ or —N(OH)R₁₃;
R₁₀ is —H or an alkyl group;
R₁₁ is —H, an alkyl group or an acyl group;
R₁₂ is —H or an alkyl group;
R₁₃ is an alkyl group,

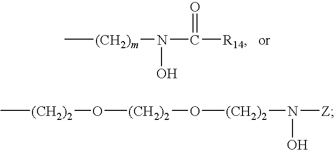

R₁₄ is an alkyl group;
R′ is an alkyl group;
m is an integer from 1 to 8;
each n is independently an integer from 1 to 8;
x is an integer from 1 to 8;
y is an integer from 0 to 8;
Z is —C(O)R₁₄,

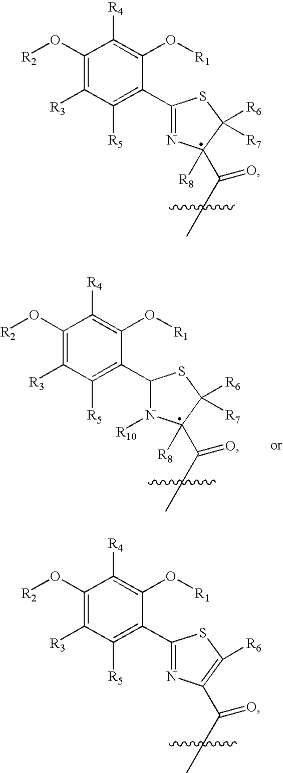

or a salt, solvate or hydrate thereof.

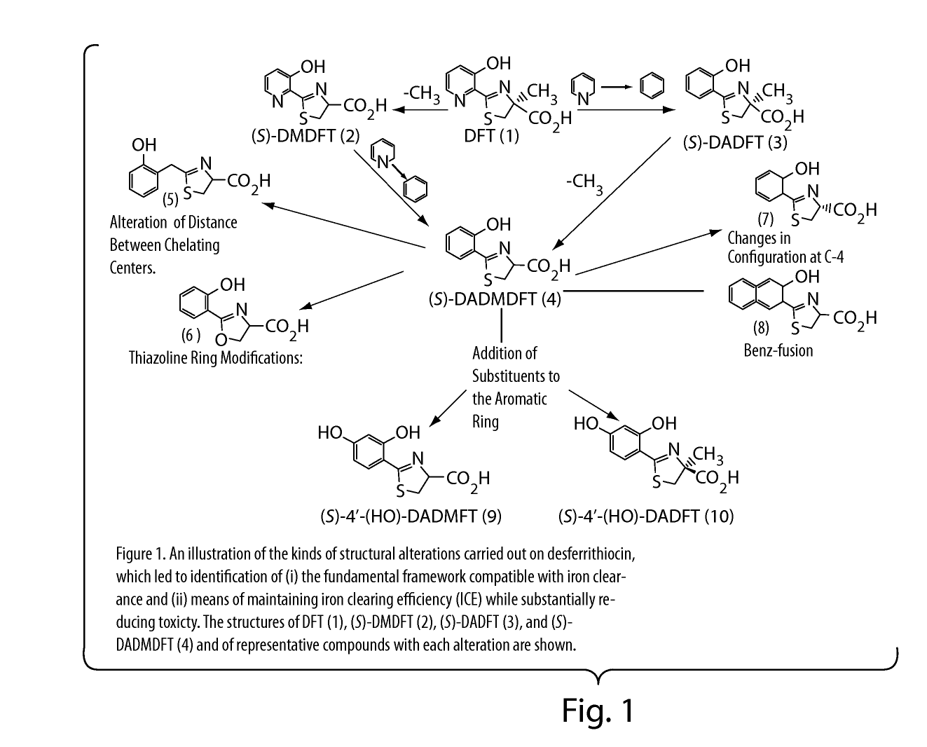

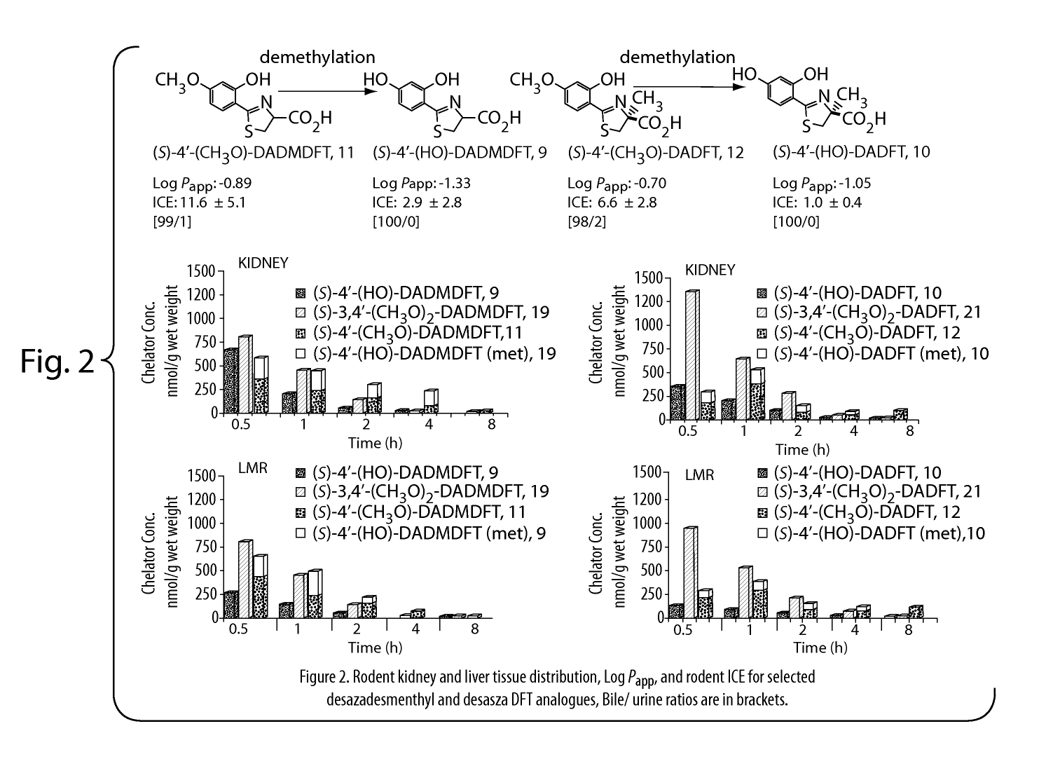

Past systematic structure-activity studies have allowed the design and synthesis of analogues and derivatives which retain the exceptional iron-chelating activity of desferrithiocin (DFT) while eliminating its adverse effects. The hypothesis underlying the present invention is that a similar approach can be adopted to utilize the DFT platform for the design of ligands that will effectively decorporate actinides. On the basis of past experience, the results of extensive studies of iron chelation in rodents and primates, and a wide-ranging review of the available scientific literature, a ligand basis set which includes a number of chelators already shown to decorporate uranium was selected to represent the best available candidates at present for the decorporation of U(VI), Th(IV) [a surrogate for Pu(IV)] and Eu(III) [a surrogate for Am(III)]. Systematic investigations in rodents (including dose-response, pharmacologic, toxicologic and histopathologic studies) identify the DFT chelators in the ligand basis set that are most effective and least toxic for decorporation of U(VI), Th(IV) and Eu(III). An innovative new approach using MRI to characterize the action of a selected chelator on distribution and elimination of Eu(III) in rodents is also utilized. Ultimately, the most promising candidate chelators are evaluated in a primate model to provide the best available evidence for efficacy in humans. Thus, the invention focuses on the design, evaluation, and development of desferrithiocin analogues for the decorporation of U(VI), Th(IV) [a surrogate for Pu(IV)], and Eu(III) [a surrogate for Am(III)] (Nash et al, Features of the thermodynamics of two-phase distribution reactions of americium(III) and europium(III) nitrates into solutions of 2,6-bis[(bis(2ethylhexyl)phosphino)-methyl]pyridine N,P,P′-trioxide. Inorg Chem 2002; 41:5849-5858) in animals. U, Pu, and Am certainly rank high as candidates for terrorist use. Six observations led to the present invention: (1) A variety of hexacoordinate ligands have been shown to bind Fe(III)[Bergeron, et al., Iron Chelators and Therapeutic Uses, Burger's Medicinal Chemistry 2003; III:479-561], PU(IV)[Jarvis, et al., Some correlations involving the stability of complexes of transuranium metal ions and ligands with negatively charged oxygen donors, Inorg Chim Acta 1991; 182:229-232; Neu, et al., Structural Characterization of a Plutonium(IV) Siderophore Complex: Single-Crystal Structure of Pu-Desferrioxamine E, Angewandte Chemie International Edition 2000; 39:1442-1444; Durbin, et al., In Vivo Chelation of Am(III), Pu(IV), Np(V), and U(VI) in Mice by TREN-(Me-3,2-HOPO), Radiat Prot Dosimetry 1994; 53:305-309], Th(IV)[Whisenhunt, et al., Specific Sequestering Agents for the Actinides. 29. Stability of the Thorium(IV) Complexes of Desferrioxamine B (DFO) and Three Octadentate Catecholate or Hydroxypyridinonate DFO Derivatives: DFOMTA, DFOCAMC, and DFO-1,2-HOPO. Comparative Stability of the Plutonium(IV) DFOMTA Complex(I), Inorg Chem 1996; 35:4128-4136; Langer, Solid complexes with tetravalent metal ions and ethylenediamime tetra-acetic acid (EDTA), J Inorg Nucl Chem 1964; 26:59-72], Am(III)[Durbin, et al., In Vivo Chelation of Am(III), Pu(IV), Np(V), and U(VI) in Mice by TREN-(Me-3,2-HOPO), Radiat Prot Dosimetry 1994; 53:305-309],Eu(III) and U(VI)[Durbin, et al., In Vivo Chelation of Am(III), Pu(IV), Np(V), and U(VI) in Mice by TREN-(Me-3,2-HOPO), Radiat Prot Dosimetry 1994; 53:305-309). (2) Analogues of desferrithiocin [(S)-4,5-dihydro-2-(3-hydroxy-2-pyridinyl)-4methyl-4-thiazolecarboxylic acid (DFT, 1, FIG. 1)] have been shown to form 2:1 hexacoordinate complexes with Fe(III) and Th(IV)[Rao, et al., Complexation of Thorium(IV) with Desmethyldesferrithiocin, Radiochim Acta 2000; 88:851-856]. (3) These same ligands, when administered either subcutaneously (SC) or orally (PO) to rodents, dogs, and primates, have been shown to clear iron very efficiently. (4) These ligands have also been shown to decorporate uranium from rodents: they are effective given intraperitoneally (IP) and SC or PO. They can have a profound effect on clearing uranium from kidneys. (5) One of these ligands (S)-2-(2,4-dihydroxyphenyl)-4,5-dihydro-4-methyl-4-thiazolecarboxylic acid [(S)-4′-(HO)-DADFT, 10, FIG. 1] is an orally effective iron chelator in human clinical trials for the treatment of patients with iron overload. This same ligand also decorporates uranium from rodents. (6) A second analogue, (S)-2-(2-hydroxy-4-methoxyphenyl)-4,5dihydro-4-thiazolecarboxylic acid [(S)-4′-(CH3O)-DADMDFT, 11, FIG. 2], is more efficient than 10 at decorporating uranium and has been through the NIH-RAID program. Based on these collective observations, we assessed the ability of a group of desferrithiocin analogues which form 2:1 hexacoordinate complexes with Fe(III) and Th(IV) to decorporate Th(IV), Eu(III), and U(VI) from animals as proof of principle.

These preliminary results (1) illustrate, in a very general way, the kind of systematic structure-activity approach [Bergeron, et al., Effects of C-4 Stereochemistry and C-4′ Hydroxylation on the Iron Clearing Efficiency and Toxicity of Desferrithiocin Analogues, J Med Chem 1999; 42:2432-2440; Bergeron, et al., The Desferrithiocin Pharmacophore, J Med Chem 1994; 37:1411-1417; Bergeron, et al., Desazadesmethyldesferrithiocin Analogues as Orally Effective Iron Chelators, J Med Chem 1999; 42:95-108; Bergeron, et al., Synthesis and Biological Evaluation of Naphthyldesferrithiocin Iron Chelators, J Med Chem 1996; 39:1575-1581] adopted to bring an orally effective iron chelator, e.g., (S)-4′-(HO)-DADFT (10, FIG. 1) to light [Donovan, et al., Preclinical and clinical development of deferitrin, a novel, orally available iron chelator, Ann N Y Acad Sci 2005; 1054:492-494] for the treatment of children with iron overload disease, ie, thalassemia, and (2) show that the desferrithiocin (DFT) platform is a good candidate for the development of new therapeutics for the decorporation of uranium, plutonium, americium, and thorium.

In the text below, “iron-clearing efficiency” (ICE) [Bergeron, et al., Effects of C-4 Stereochemistry and C-4′ Hydroxylation on the Iron Clearing Efficiency and Toxicity of Desferrithiocin Analogues, J Med Chem 1999; 42:2432-2440] is used as a measure of the amount of iron excretion induced by a chelator. The ICE, expressed as a percent, is calculated as (ligand-induced iron excretion/theoretical iron excretion)×100. To illustrate, the theoretical iron excretion after administration of one millimole of desferrioxamine (DFO, 14, Table 1), a hexadentate chelator which forms a 1:1 complex with Fe(III), is one milli-g-atom of iron. Two millimoles of desferrithiocin (DFT, 1, FIG. 1), a tridentate chelator which forms a 2:1 complex with Fe(III), are required for the theoretical excretion of one milli-g-atom of iron. In clinical practice, the actual ICE of DFO (14), used to treat people with iron overload, is only 5 to 7% [Pippard, et al., Iron Chelation Using Subcutaneous Infusions of Diethylene Triamine Penta-acetic Acid (DTPA), Scand J Haematol 1986; 36:466-472; Pippard, Desferrioxamine-Induced Iron Excretion in Humans, Bailliere's Clin Haematol 1989; 2:323-343]. After subcutaneous administration of 14 to the iron-loaded Cebus apella primate, the ICE is virtually identical to that found in patients, 5.0±2.6% [Bergeron, et al., A Comparative Study of the Iron-Clearing Properties of Desferrithiocin Analogues with Desferrioxamine B in a Cebus Monkey Model, Blood 1993; 81:2166-21731 The lead DFT analogue now in clinical trials, (S)-4′-(HO)-DADFT (10, FIG. 1), given orally to the primates at an iron binding equivalent dose, has an ICE that is nearly three times (13.4±5.8%) that of parenterally administered DFO [Bergeron, et al., Structure-Activity Relationships Among Desazadesferrithiocin Analogues, Adv Exp Med Biol 2002; 509:167-184]. Structure-Activity Relationships (SARs) among Tridentate DFT Analogues, DFT (1, FIG. 1) is a tridentate siderophore [Naegeli, et al., Metabolites of Microorganisms. Part 193. Ferrithiocin, Hely Chim Acta 1980; 63:1400-1406] that forms a stable 2:1 complex with Fe(III); the cumulative formation constant is 4×1029 M−1 [Hahn, et al., Coordination Chemistry of Microbial Iron Transport. 42. Structural and Spectroscopic Characterization of Diastereomeric Cr(III) and Co(III) Complexes of Desferriferrithiocin., J Am Chem Soc 1990; 112:1854-1860; Anderegg, et al., Metal Complex Formation of a New Siderophore Desferrithiocin and of Three Related Ligands, J Chem Soc, Chem Commun 1990; 1194-1196]. The donor groups include a phenolic oxygen, a thiazoline nitrogen, and a carboxyl. DFT (1) was one of the first iron chelators shown to be orally active [Wolfe, et al., A Non-Human Primate Model for the Study of Oral Iron Chelators, Br J Haematol 1989; 72:456-461]. It performed well in both the bile duct-cannulated rodent model (ICE, 5.5%) [Bergeron, et al., Evaluation of Desferrithiocin and Its Synthetic Analogues as Orally Effective Iron Chelators, J Med Chem 1991; 34:2072-2078] and in the iron-overloaded C. apella primate (ICE, 16%) [Bergeron, et al., A Comparative Study of the Iron-Clearing Properties of Desferrithiocin Analogues with Desferrioxamine B in a Cebus Monkey Model, Blood 1993; 81:2166-2173; Bergeron, et al., A Comparative Evaluation of Iron Clearance Models, Ann N Y Acad Sci 1990; 612:378-393]. Unfortunately, DFT (1) is severely nephrotoxic [Bergeron, et al., A Comparative Study of the Iron-Clearing Properties of Desferrithiocin Analogues with Desferrioxamine B in a Cebus Monkey Model, Blood 1993; 81:2166-2173]. Nevertheless, the outstanding oral activity spurred a structure-activity study to identify an orally active and safe DFT analogue. The first goal was to define the minimal structural platform, i.e., pharmacophore, compatible with iron clearance upon PO administration (FIG. 1).

Our initial approach entailed simplifying the platform. The thiazoline methyl of DFT (1) was deleted to produce (S)-4,5-dihydro-2-(3-hydroxy-2-pyridinyl)-4-thiazolecarboxylic acid [desmethyldesferrithiocin, (S)-DMDFT, 2, FIG. 1], reducing the ICE by two-thirds from 16% to 4.8% in the primate model [Bergeron, et al., A Comparative Study of the Iron-Clearing Properties of Desferrithiocin Analogues with Desferrioxamine B in a Cebus Monkey Model, Blood 1993; 81:2166-2173]. Removal of DFT's aromatic nitrogen left (S)-4,5-dihydro-2-(2-hydroxyphenyl)-4-methyl-4-thiazolecarboxylic acid [desazadesferrithiocin, (S)-DADFT, 3], modestly diminishing the compound's ICE to 13% in C. paella [Bergeron, et al., Effects of C-4 Stereochemistry and C-4′ Hydroxylation on the Iron Clearing Efficiency and Toxicity of Desferrithiocin Analogues, J Med Chem 1999; 42:2432-2440]. Abstraction of the thiazoline methyl from (S)-DADFT, leaving (S)-4,5-dihydro-2-(2-hydroxyphenyl)-4-thiazolecarboxylic acid [desazadesmethyldesferrithiocin, (S)-DADMDFT, 4], had little effect on efficacy, 12% vs 13% ICE [Bergeron, et al., Effects of C-4 Stereochemistry and C-4′ Hydroxylation on the Iron Clearing Efficiency and Toxicity of Desferrithiocin Analogues, J Med Chem 1999; 42:2432-2440; Bergeron, et al., A Comparative Study of the Iron-Clearing Properties of Desferrithiocin Analogues with Desferrioxamine B in a Cebus Monkey Model, Blood 1993; 81:2166-2173]. These observations suggested that more apparently lipophilic chelators are more active, eg, 1, 3, or 4 vs 2.

We have now confirmed this idea [Bergeron, et al., Impact of the Lipophilicity of Desferrithiocin Analogues on Iron Clearance, Medicinal Inorganic Chemistry 2005; 366-383; Bergeron, et al., Partition-Variant Desferrithiocin Analogues: Organ Targeting and Increased Iron Clearance, J Med Chem 2005; 48:821-831]. Few further structural changes could be made to the (S)-DADMDFT (4, FIG. 1) framework without loss of activity in the C. apella model. Alterations of the distances between the donor centers, e.g., 5, resulted in loss of activity[Bergeron, et al., Desazadesmethyldesferrithiocin Analogues as Orally Effective Iron Chelators, J Med Chem 1999; 42:95-108]. Thiazoline ring modifications, i.e., expansion (dihydrothiazine), oxidation (thiazole), or reduction (thiazolidine), abrogated iron-clearing activity [Bergeron, et al., The Desferrithiocin Pharmacophore, J Med Chem 1994; 37:1411-1417; Bergeron, et al., Desazadesmethyldesferrithiocin Analogues as Orally Effective Iron Chelators, J Med Chem 1999; 42:95-108]. Likewise, replacement of the sulfur with oxygen (oxazolines, eg, 6), with nitrogen (dihydroimidazole), or with a methylene (dihydropyrrole) resulted in significant loss of efficacy. [Bergeron, et al., The Desferrithiocin Pharmacophore, J Med Chem 1994; 37:141 1-1417; Bergeron, et al., Desazadesmethyldesferrithiocin Analogues as Orally Effective Iron Chelators, J Med Chem 1999; 42:95-108.] Changes in configuration at C-4 also had a profound effect on ICE [Bergeron, et al., Effects of C-4 Stereochemistry and C-4′ Hydroxylation on the Iron Clearing Efficiency and Toxicity of Desferrithiocin Analogues, J Med Chem 1999; 42:2432-2440; Bergeron, et al., Synthesis and Biological Evaluation of Naphthyldesferrithiocin Iron Chelators, J Med Chem 1996; 39:1575-1581; Bergeron, et al., Iron Chelation Promoted by Desazadesferrithiocin Analogues: An Enantioselective Barrier, Chirality 2003; 15:593-599], demonstrating a potential stereoselective barrier in iron clearance: (S)-enantiomers are always more active in the primates than (R)-enantiomers, e.g., 7.[Bergeron, et al., Iron Chelation Promoted by Desazadesferrithiocin Analogues: An Enantioselective Barrier, Chirality 2003; 15:593-599; Bergeron, et al., The Origin of the Differences in (R)- and (S)-Desmethyldesferrithiocin: Iron-Clearing Properties, Ann N Y Acad Sci 1998; 850:202-216.] Benz-fusions, designed to improve the ligands' tissue residence time and possibly ICE, were ineffective; both the naphthyl analogues, e.g., 8, and the quinoline systems performed poorly. [Bergeron, et al., Desazadesmethyldesferrithiocin Analogues as Orally Effective Iron Chelators, J Med Chem 1999; 42:95-108; Bergeron, et al., Synthesis and Biological Evaluation of Naphthyldesferrithiocin Iron Chelators, J Med Chem 1996; 39:1575-1581.] Having identified the simplest framework (4), the issue then became one of reducing toxicity.

Both DADFT analogues, 3 and 4, are still quite toxic. Severe gastrointestinal (GI) toxicity was prominent, rather than nephrotoxicity as with DFT.[Bergeron, et al., Effects of C-4 Stereochemistry and C-4′ Hydroxylation on the Iron Clearing Efficiency and Toxicity of Desferrithiocin Analogues, J Med Chem 1999; 42:2432-2440; Bergeron, et al., Desazadesmethyldesferrithiocin Analogues as Orally Effective Iron Chelators, J Med Chem 1999; 42:95-108; Bergeron, et al., A Comparative Study of the Iron-Clearing Properties of Desferrithiocin Analogues with Desferrioxamine B in a Cebus Monkey Model, Blood 1993; 81:2166-2173.] The (S)-DADMDFT (4, FIG. 1) framework was then subjected to a structure-activity study aimed at ameliorating its toxicity. This structure-activity approach was based on the idea that by altering the lipophilicity (i.e., partition properties, log Papp) and/or redox potential, the drug's organ distribution properties, metabolic disposition, and toxicity profile could change.

This was accomplished by addition of aromatic ring substituents and/or the presence or absence of the thiazoline methyl [Bergeron, et al., Desazadesmethyldesferrithiocin Analogues as Orally Effective Iron Chelators, J Med Chem 1999; 42:95-108.] Ultimately we discovered that addition of electron-donating groups, as in the systems (S)-2(2,4-dihydroxyphenyl)-4,5-dihydro-4-thiazolecarboxylic acid [(S)-4′-(HO)-DADMDFT, 9, FIG. 1] and (S)-4′(HO)-DADFT (10) was compatible with iron clearance in the primate model [Bergeron, et al., Effects of C-4 Stereochemistry and C-4′ Hydroxylation on the Iron Clearing Efficiency and Toxicity of Desferrithiocin Analogues, J Med Chem 1999; 42:2432-2440; Bergeron, et al., Desazadesmethyldesferrithiocin Analogues as Orally Effective Iron Chelators, J Med Chem 1999; 42:95-108; Bergeron, et al., Methoxylation of Desazadesferrithiocin Analogues: Enhanced Iron Clearing Efficiency, J Med Chem 2003; 46:1470-1477.] This hydroxylation profoundly diminished the toxicity of the resulting derivatives. For example, rats that were treated with 3 or 4 were dead by day 5 of a planned 10-day dosing regimen [Bergeron, et al., A Comparative Study of the Iron-Clearing Properties of Desferrithiocin Analogues with Desferrioxamine B in a Cebus Monkey Model, Blood 1993; 81:2166-2173]; those administered (S)-4′-(HO)-DADMDFT (9) and (S)-4′-(HO)-DADFT (10) at the same dose did not display any frank toxicity [Bergeron, et al., Effects of C-4 Stereochemistry and C-4′ Hydroxylation on the Iron Clearing Efficiency and Toxicity of Desferrithiocin Analogues, J Med Chem 1999; 42:2432-2440.] In fact, (S)-4′-(HO)-DADFT (10) is now the lead compound in clinical trials. Whereas ring hydroxylation can decrease the ICE of the parent drug, e.g., a 5.3% ICE for 9 [vs 12.4% for 4 when administered PO at a dose of 150 μmol/kg], in the case of (S)DADFT (3) and (S)-4′-(HO)-DADFT (10), the ICEs were nearly identical.

In summary, we have successfully taken a natural product iron chelator, DFT, shown to form 2:1 hexacoordinate complexes with Fe{III), which, while effective at removing iron, was profoundly nephrotoxic, and through structure-activity studies assembled an equally active, nontoxic, orally effective analogue (now in clinical trials) for the treatment of iron overload disease, i.e., thalassemia. Recall that a number of hexacoordinate ligands have been shown to decorporate Th(IV), Pu(IV), Am(III), and U(VI) from animals [Durbin, et al., In Vivo Chelation of Am(III), Pu(IV), Np(V), and U(VI) in Mice by TREN-(Me-3,2-HOPO), Radiat Prot Dosimetry 1994; 53:305-309; Gorden, et al., Rational design of sequestering agents for plutonium and other actinides, Chem Rev 2003; 103:4207-4282; Durbin, et al., Chelating agents for uranium(VI): 2. Efficacy and toxicity of tetradentate catecholate and hydroxypyridinonate ligands in mice, Health Phys 2000; 78:511-521; Guilmette, et al., Competitive binding of Pu and Am with bone mineral and novel chelating agents, Radiat Prot Dosimetry 2003; 105:527-534.]

Tissue Distribution of DFT Analogues. While in the design strategies of chelators for the treatment of iron overload disease the major organs of concern are the liver, pancreas, and heart, the therapeutic targets for the decorporation of uranium, plutonium, americium and thorium are the kidney, liver, lung and bone [Gorden, et al., Rational design of sequestering agents for plutonium and other actinides, Chem Rev 2003; 103:4207-4282; Luciani, et al., Americium in the beagle dog: biokinetic and dosimetric model, Health Phys 2006; 90:459-470.] In structure-activity studies with DFT analogues, it became clear that they can have profoundly different organ distribution and tissue residence times that are often tied to their lipophilicity, log Papp. FIG. 2 illustrates the disposition of two different families of ligands in kidney and liver tissue [Bergeron, et al., (S)-4,5-Dihydro-2-(2-hydroxy-4-hydroxyphenyl)-4-methyl-4-thiazolecarboxylic Acid Polyethers: A Solution to Nephrotoxicity, J Med Chem 2006; 49:2772-2783.] Note that (S)-4′(CH3O)-DADMDFT (11) and the corresponding (S)-4,5-dihydro2-(2-hydroxy-4-methoxyphenyl)4-methyl-4-thiazolecarboxylic acid [(S)-4′-(CH3O)-DADFT, 12] are both metabolically O-demethylated to (S)-4′-(HO)-DADMDFT (9) and (S)-4′-(HO)-DADFT (10), respectively. Both metabolites are also active iron chelators. The renal and hepatic distribution of (S)-4,5-dihydro-2(3,4-dimethoxy-2-hydroxyphenyl)-4-thiazolecarboxylic acid [(S)-3′,4′-(CH3O)-DADMDFT, 19, Table 2)] and the corresponding (S)-4,5-dihydro-2-(3,4-dimethoxy-2-hydroxyphenyl)-4-methyl-4-thiazolecarboxylic acid [(S)-3′,4′-(CH3O)-DADFT, 21, Table 2)] are also shown. Similar differences in disposition were seen in the pancreas and heart. These ligands represent a very limited example of the many analogues evaluated for tissue distribution.

Chelator Access to Lung Tissue. As inhalation is one of the principal routes of potential actinide contamination, in a preliminary study aimed at this proposal, the accumulation of two different DFT analogues in lung tissue, (S)-4′-(HO)-DADMDFT (9, FIG. 1) and (S)-3′,4′-(CH3O)-DADMDFT, 19, Table 2)] was investigated. Rodents were given the drugs SC at a dose of 300 μmol/kg. The latter chelator is far more lipophilic than the former. The more lipophilic chelator (19) achieved a concentration of 290±66 nmol/g wet weight 0.5 h post drug. The level of the less lipophilic ligand (9) was much lower, 80±9 nmol/g wet weight 0.5 h post drug. This is consistent with the idea that organ targeting can be achieved,[Bergeron, et al., Impact of the Lipophilicity of Desferrithiocin Analogues on Iron Clearance, Medicinal Inorganic Chemistry 2005; 366-383] corroborating previous studies and further contributing to the index of success of the invention.

Desferrithiocin Analogue Induced Excretion of Uranium. In order to compare results with previous chelator-induced iron excretion data, the uranium clearance studies were carried out in a bile duct-cannulated rat model. The animals were given uranyl acetate dihydrate SC at a dose of 5 mg/kg (the actual dose of uranium is 2.8 mg/kg). The chelators were given IP, SC or PO at times relative to uranium exposure, indicated in Table 1. Bile and urine samples were collected for 24 hours after dosing. Kidneys were removed from selected animals and tissue uranium concentration measured. At least three animals were utilized in each experimental group. Data from two separate control studies (uranyl acetate/ no chelator) have now been combined for a total of 14 control animals. All uranium concentrations were measured using ICPMS. The data are reported as the total quantity of uranium excreted [urine +bile]; the mode of excretion [urine/bile] is also given. In addition the percentage of the administered dose of uranium cleared and chelator-induced uranium excretion vs the controls is also given. Four positive controls were evaluated: DTPA (13, Table 1) given as its trisodium calcium salt, DFO (14), N,N′-bis(2hydroxybenzyl)ethylenediamine-N,N′-diacetic acid, monosodium salt (NaHBED, 15) and the hydroxypyridone CP94 (16) shown to bind uranium [Pashalidis, et al., Effective complex formation in the interaction of 1,2-dimethyl-3-hydroxypyrid-4-one (Deferiprone or L1) with uranium(VI), Journal of Radioanalytical and Nuclear Chemistry 1999; 242:181-184.] The total metal cleared after 24 h in control rats was 10% of the administered dose. When DTPA, 13, was given IP or PO at 300 μmol/kg immediately after uranium, the excretion was 17% and 8% of the administered dose respectively (p>0.05). However, when the drug was given IP at a dose of 600 μmol/kg immediately post-metal, the uranium excretion was significant, 20% (p<0.005). This is consistent with previously published data using a similar animal model.[Domingo, et al., Comparative effects of the chelators sodium 4,5-dihydroxybenzene-1,3-disulfonate (Tiron) and diethylenetriaminepentaacetic acid (DTPA) on acute uranium nephrotoxicity in rats, Toxicology 1997; 118:49-59] DFO (14) and CP94 (16) given IP immediately post uranyl acetate were ineffective. NaHBED (15) marginally improved clearance (Table 1).

The scenario was very different with the DFT analogues. When (S)-4′-(CH3O)-DADFT (12) was given IP at 300 μmol/kg immediately after the uranium, metal excretion increased to 20% (p<0.003), but dropped to within error of control when given 0.5 h post metal. With (S)-4,5-dihydro-5,5-dimethyl-2-(2-hydroxyphenyl)-4-thiazolecarboxylic acid [(S)-5,5-(CH3O)-DADMDFT, 17] given IP immediately post uranium, the excretion was 26% (p<0.002), but was again insignificant when given 0.5 h post metal. (S)-4′-(HO)-DADFT (10), given IP immediately post metal, increased uranium excretion to 19% (p<0.05), while the corresponding polyether, (S)-4,5-dihydro-2-[2-hydroxy-4-(3,6,9-trioxadecyloxy)phenyl]-4methyl-4-thiazolecarboxylic acid [(S)-4′-(HO)-DADFT-PE (18)], given IP immediately post metal, raised uranium excretion to 22% (p<0.001, Table 1).

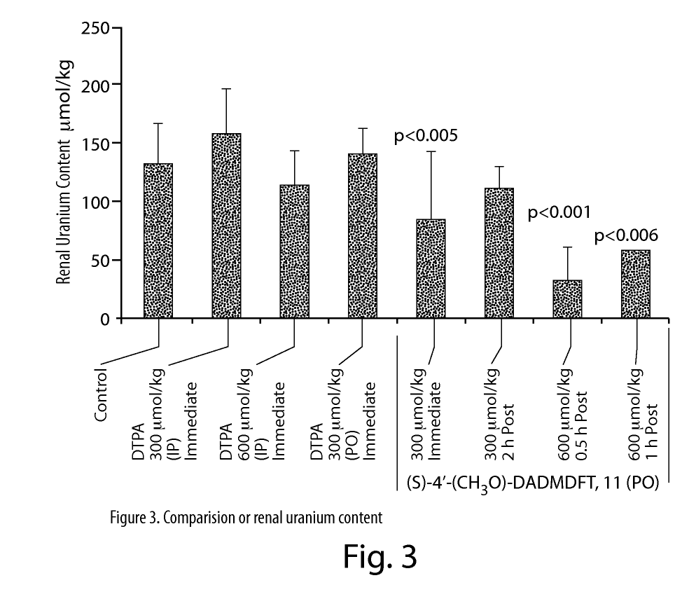

The most promising analogue in the (S)-2-(2,4-dihydroxyphenyl)-4,5-dihydro-4-thiazolinecarboxylic series is the methoxy analogue (S)-4′-(CH3O)-DADMDFT (11). Given IP at 300 μmol/kg 0.5 h before uranyl acetate or immediately thereafter, nearly 30% of the metal was cleared (Table 1). This was also true if the drug was given IP 0.5 or 2 h post metal. The drug retains some efficacy given 4 h post uranium; 16% of the metal is excreted (p<0.006). When the chelator was given SC immediately post uranium, 40% of the administered metal was cleared. The increased excretion is probably associated with a slower absorption of the ligand. The data from rodents given the drug orally is even more encouraging (Table 1). When the chelator was administered PO at a dose of 300 μmol/kg immediately post metal, clearance was 23% (p<0.001), indicating very good oral bioavailability. At 2 h post metal the clearance was still significant, 18%, (p<0.005). When the dose of the chelator was increased to 600 μmol/kg and given PO 0.5 or 1 h post uranium, the clearance was 25% (p<0.001) and 26% (p<0.006) respectively (Table 1). The most profound data are associated with uranium decorporation from the kidneys (FIG. 3). When DTPA is given IP or PO at a dose of 300 μmol/kg immediately post metal, there is no reduction in renal uranium relative to controls. However, (S)-4′-(CH3O)-DADMDFT (11) given orally at a dose of 300 μmol/kg immediately post metal reduces renal uranium by 37% (p<0.005).

There is a small (16%) reduction when the drug is given PO at a dose of 300 μmol/kg 2 h post uranium. However, when 11 is given PO at a dose of 600 μmol/kg 0.5 h post metal, the renal uranium content is reduced by 76% (p<0.001). When given PO at the same dose 1 h post uranium exposure, the reduction is still significant, 42% (p<0.006). In all sets of ligands, the most lipophilic chelator is always the most toxic. It was recently demonstrated that it is possible to design ligands that balance the lipophilicity/toxicity problem while iron-clearing efficiency is maintained. Earlier studies with (S)-4′-(CH3O)-DADFT (12, Table 1)[Bergeron, et al., Partition-Variant Desferrithiocin Analogues: Organ Targeting and Increased Iron Clearance, J Med Chem 2005; 48:821-831] indicated that this methyl ether was a ligand with excellent iron-clearing efficiency in both rodents and primates; however, it was too toxic for clinical consideration. On the basis of this finding, a less lipophilic, more water-soluble ligand than 12 was assembled, (S)-4′-(HO)-DADFT-PE (18, Table 1), a polyether analogue. The polyether was shown to be a highly efficient iron chelator in both rodents and primates.

The dose limiting toxicity of (S)-4′-(HO)-DADFT (10, Table 1) will likely be renal toxicity. A comparison of 18 in rodents with 10 revealed the polyether to be more tolerable, achieving higher concentrations in the liver and significantly lower concentrations in the kidney. The lower renal drug levels are in keeping with the profound difference in the architectural changes seen in the kidneys of rodents given 10 versus those treated with 18,[Bergeron, et al., (S)-4,5-Dihydro-2-(2-hydroxy-4-hydroxyphenyl)-4-methyl-4-thiazolecarboxylic Acid Polyethers: A Solution to Nephrotoxicity, J Med Chem 2006; 49:2772-2783.] This same polyether is also active at clearing uranium (Table 1).

Iron VS. Actinide Decorporation. All of the desferrithiocins can be expected to clear iron from animals. This elicits two questions: (1) Will competition of the ligands for iron vs actinides be a problem? (2) Will protracted exposure of humans to such a chelator deplete enough iron to cause untoward effects?

In all previous studies with the ligands given to iron-overloaded primates SC or PO, the ICE is less than or equal to 25%. Thus 75% of the chelator is available for actinide decorporation. Furthermore, the uranium studies unequivocally show the metal competition issue is not a problem. While protracted exposure to the chelator, if required, could cause iron removal problems, iron is easily replaced clinically.

Preliminary Data Overview.

1. The synthetic chemistry for the assembly of a wide variety of DFT analogues is in place and lends itself nicely to industrial scale-up[Bergeron, et al., Effects of C-4 Stereochemistry and C-4′ Hydroxylation on the Iron Clearing Efficiency and Toxicity of Desferrithiocin Analogues, J Med Chem 1999; 42:2432-2440; Bergeron, et al., Desazadesmethyldesferrithiocin Analogues as Orally Effective Iron Chelators, J Med Chem 1999; 42:95-108; Bergeron, et al., Synthesis and Biological Evaluation of Naphthyldesferrithiocin Iron Chelators, J Med Chem 1996; 39:1575-1581; Bergeron, et al., Partition-Variant Desferrithiocin Analogues: Organ Targeting and Increased Iron Clearance, J Med Chem 2005; 48:821-831; Bergeron, et al., Methoxylation of Desazadesferrithiocin Analogues: Enhanced Iron Clearing Efficiency, J Med Chem 2003; 46:1470-1477; Bergeron, et al., (S)-4,5-Dihydro-2-(2-hydroxy-4-hydroxyphenyl)-4-methyl-4-thiazolecarboxylic Acid Polyethers: A Solution to Nephrotoxicity, J Med Chem 2006; 49:2772-2783; Bergeron, et al., Desferrithiocin Analogue-Based Hexacoordinate Iron((III)) Chelators, J Med Chem 2003; 46:16-24.]

2. Structure-activity studies have defined the physiochemical properties of the DFT analogues which control toxicity and tissue distribution [Bergeron, et al., Partition-Variant Desferrithiocin Analogues: Organ Targeting and Increased Iron Clearance, J Med Chem 2005; 48:821-831; Bergeron, et al., (S)-4,5-Dihydro-2-(2-hydroxy-4-hydroxyphenyl)-4-methyl-4-thiazolecarboxylic Acid Polyethers: A Solution to Nephrotoxicity, J Med Chem 2006; 49:2772-2783.]

3 Analytical methods are in place for following tissue distribution and pharmacokinetics of the analogues[Bergeron, et al., (S)-4,5-Dihydro-2-(2-hydroxy-4-hydroxyphenyl)-4-methyl-4-thiazolecarboxylic Acid Polyethers: A Solution to Nephrotoxicity, J Med Chem 2006; 49:2772-2783; Bergeron, et al., Pharmacokinetics of Orally Administered Desferrithiocin Analogs in Cebus apella Primates, Drug Metab Dispos 1999; 27:1496-1498.]

4. An ICPMS system is in place and all of the appropriate assay conditions have been worked out.

5. A number of the DFT analogues investigated also clear uranium in a rodent model. One of these analogues (S)-4′-(HO)-DADFT (10, FIG. 1 and Table 1)[Bergeron, et al., Effects of C-4 Stereochemistry and C-4′ Hydroxylation on the Iron Clearing Efficiency and Toxicity of Desferrithiocin Analogues, J Med Chem 1999; 42:2432-2440] is an orally active iron chelator currently in human trials for the treatment of iron overload disease[Donovan, et al., Preclinical and clinical development of deferitrin, a novel, orally available iron chelator, Ann N Y Acad Sci 2005; 1054:492-494.]

6. A second chelator, (S)-4′-(CH3O)-DADMDFT (11, FIG. 2 and Table 1), works well as a decorporation agent dosed IP, SC, or PO and clears a profound amount of the metal from the kidney after a single oral exposure.

7. This same ligand (11) has now been run through the NIH Roadmap Program and a complete GLP toxicity and pharmacokinetics profile is available. The no-observed-adverse-effect-level is well within the range of expected human dosing requirements.

8. A long history in the design, synthesis, and testing of chelators; rodent and primate models are all in place [Bergeron, et al., Iron Chelators and Therapeutic Uses, Burger's Medicinal Chemistry 2003; III:479-561; Bergeron, et al., Desazadesmethyldesferrithiocin Analogues as Orally Effective Iron Chelators, J Med Chem 1999; 42:95-108; Bergeron, et al., A Comparative Study of the Iron-Clearing Properties of Desferrithiocin Analogues with Desferrioxamine B in a Cebus Monkey Model, Blood 1993; 81:2166-2173; Bergeron, et al., Metabolism and Pharmacokinetics of N¹,N¹⁴-Diethylhomospermine, Drug Metab Dispos 1996; 24:334-343; Bergeron, et al., A Comparison of Iron Chelator Efficacy In Iron-Overloaded. Beagle Dogs and Monkeys (Cebus apella), Comp Med 2004; 54:664-672.]

The invention focuses on the design, evaluation and development of desferrithiocin analogues for the decorporation of U(VI), Th(IV) and Eu(III), as well as other actinides. The ligand basis set (Table 2) was chosen predicated on earlier iron clearance studies in rats and primates. There are two families of chelators, the cysteine-derived compounds 9, 11, 19, 20 and alpha-methyl cysteine-derived ligands 10, 12, 18, 21. The intent is to establish a structure-activity relationship in animal models which enables the design of actinide clearing ligands. These two families have different physicochemical properties and pharmacological profiles. The alpha-methylated ligands are more lipophilic, e.g., (S)-4′-(HO)-DADMDFT (9), log Papp=−1.33 vs (S)-4′-(HO)-DADFT (10), log Papp=−1.05. The more lipophilic methyl cysteine systems usually have higher iron-clearing efficiencies and different organ distribution patterns than the less lipophilic cysteine systems.

Most of the ligands in Table 2 have been evaluated for their iron-clearing efficiency in rodents and primates 9-12, 18, 19, log Papp (lipophilicity) 9-12, 18, 19, 21, and in several cases as uranium decorporation agents after IP dosing 10-12, 18 or SC and PO administration (11).

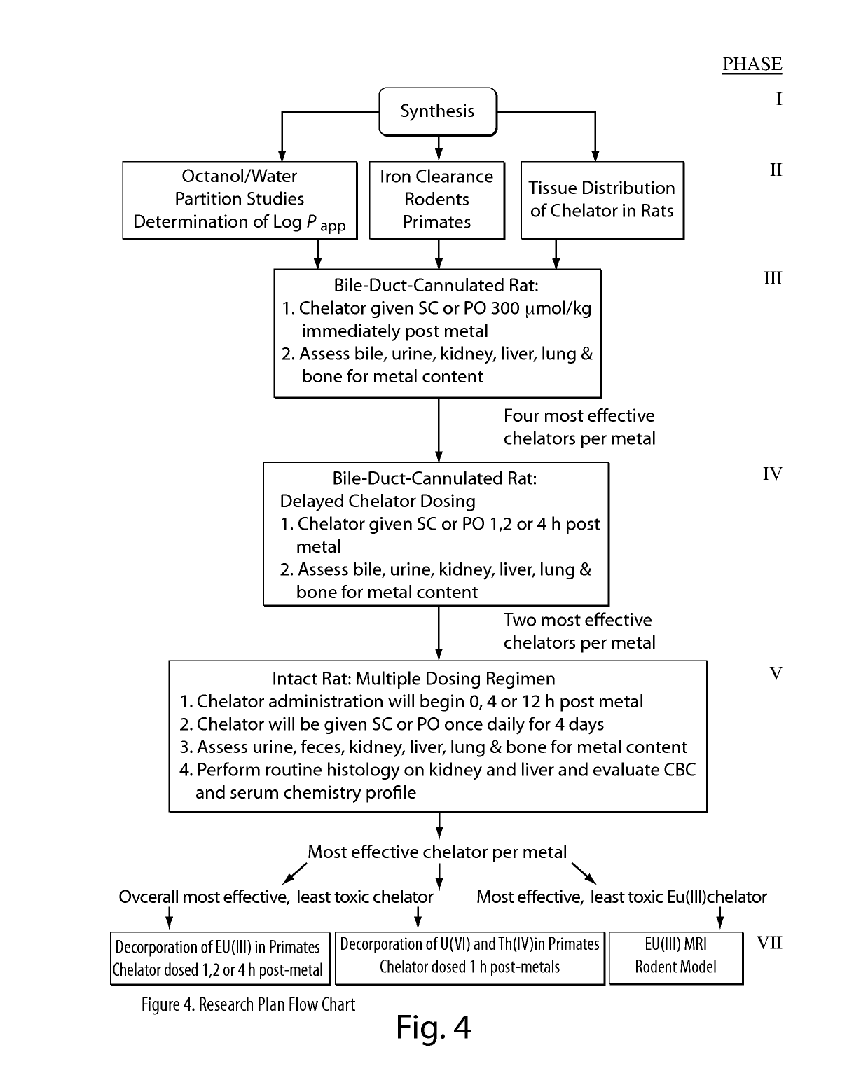

Organ/Tissue Distribution. All of the ligands are evaluated for distribution to lung and bone (femur) (Phase II, FIG. 4). Male Sprague-Dawley rats are given a single 300 μmol/kg dose of the chelator of interest PO and SC. The animals (n=3/route/time point) are sacrificed via exposure to CO2 gas at 0.5, 1, 2, 4 and 8 h post-drug. Blood is collected to allow for determination of the pharmacokinetics of the ligand. Lung and bone are assessed for their chelator content 9-12, 18, 21 [Bergeron, et al., (S)-4,5-Dihydro-2-(2-hydroxy-4-hydroxyphenyl)-4-methyl-4-thiazolecarboxylic Acid Polyethers: A Solution to Nephrotoxicity, J Med Chem 2006; 49:2772-2783; Bergeron, et al., Pharmacokinetics of Orally Administered Desferrithiocin Analogs in Cebus apella Primates, Drug Metab Dispos 1999; 27:1496-1498.]

Decorpooration of U(VI), Th(IV), and Eu(III) in Rodents. Four of the ligands 10-12, 18 have been evaluated for their ability to chelate uranium in a rodent model after IP injection. Since IP dosing is not realistic in a battlefield or other mass casualty scenario, the chelators are evaluated in the rodents using PO or SC administration 9-12, 18-21. While an orally active actinide chelator would be the therapeutic of choice, the SC metal clearing efficiency of such a ligand should also be assessed. Experience to date with iron chelators and uranium decorporation agents, e.g., 11 (Table 1), has shown that even when oral activity is good, SC administration can produce substantively better results. In a mass exposure scenario first responders would initiate PO dosing and might later continue with SC administration using U(VI) decorporation studies with the compounds in which there already exists ICE 9-12,18,19 and log Papp data 9-12,18,19,21. Decorporation of Th(IV) and Eu(III) is initiated in a bile duct-cannulated rodent model with the same starting set 9-12,18-21. The experimental roadmap is as described below and outlined in FIG. 4.

All eight of the DFT analogues in Table 2 are assessed for their ability to clear U(VI) [uranyl acetate dihydrate], Th(IV) [thorium tetrachloride] and Eu(III) [europium trichloride] in a bile duct-cannulated rat model (Phase III). The rats are given a single SC injection (left shoulder) of one of the metals. The dose of Th(IV) and Eu(III) is equivalent to the 2.8 mg/kg of uranium metal previously used, e.g., 2.7 mg/kg of Th(IV) and 1.8 mg/kg of Eu(III). The chelators are given to the animals PO or SC (right hip) at a dose of 300 μmol/kg immediately post-metal. Bile and urine samples are collected for 24 h. The actinide content of the bile, urine, kidney, liver, lung and bone (femur) are determined. To be considered effective, the chelators must clear a minimum of twice the metal excreted by the metal only treated rats. DTPA serves as a positive control. The four most active decorporation agents per metal are subjected to further evaluations (Phase IV).

The goal of Phase IV is to determine if drugs deemed effective in Phase III will retain their decorporation properties if the time between metal dosing and chelator administration is increased. The four most effective ligands per metal are given PO or SC to bile duct cannulated rats at a dose of 300 μmol/kg 1, 2 or 4 hours post metal. Bile and urine samples are collected for 24 h. The actinide content of the bile, urine, kidney, liver, lung and bone (femur) are determined. In each case, progression to a longer time interval will depend on the decorporation of a minimum of twice the metal excreted by the metal only treated rats. Once again DTPA serves as a positive control. The ligands which are still active the longest time post-metal exposure are deemed the most effective chelators. The two most effective chelators per metal are assessed under a four-day dosing regimen (Phase V).

The purpose of Phase V is to assess whether or not continued dosing of the chelators results in increased metal excretion These experiments are carried out in rats that have not had their bile duct cannulated. The animals are housed in metabolic cages. Urine and feces are collected at 24-h intervals. The actinide is given SC. The two most effective ligands per metal from Phase IV are given to the rats PO or SC once daily for four days. The initial dose of the ligands are either given immediately post-metal or not until 4 or 12 h thereafter. Additional doses of the chelator are given once daily for three more days. One day post last dose the animals are sacrificed and the metal content of the urine, feces, kidney, liver, lung and bone are determined. In each case, progression to a longer time interval, e.g., 4 or 12 h, depends on the decorporation of a minimum of twice the metal excreted by the metal only treated rats. DTPA serves as a positive control. Histopathology is run on kidney and liver samples from these animals to determine if actinide-induced renal or hepatotoxicity has been prevented.

The most effective chelator per metal is subjected to a dose response study in a bile duct-cannulated rodent at 75 and 150 μmol/kg of ligand PO and SC (Phase VI). Data at 300 μmol/kg is already available.

The choice of ligand will be predicated on the efficiency with which the ligand reduces overall metal burden and how it removes metal from the kidney, liver, lung and bone. The animals are given the chelator immediately post-metal exposure. The best chelator per metal is taken through toxicity trials in rodents (Phase VI). As with the development of iron-clearing drugs, 30 day toxicity trials are run (Phase VI). The drugs are given once daily PO or SC at a dose of 1, 3 and 5 times the dose required to clear a minimum of twice the metal excreted by the metal only treated rodents. Animals are sacrificed 24 h post last dose. Routine histopathology is carried out.

The pharmacokinetics of the three best chelators will be determined in male Cebus apella monkeys as previously described [Bergeron, et al., The Origin of the Differences in (R)- and (S)-Desmethyldesferrithiocin: Iron-Clearing Properties, Ann N Y Acad Sci 1998; 850:202-216; Bergeron, et al., Pharmacokinetics of Orally Administered Desferrithiocin Analogs in Cebus apella Primates, Drug Metab Dispos 1999; 27:1496-1498](Phase VI).

MRI studies of europium distribution after intratracheal or intravenous administration. These studies develop and apply a new method for evaluating the effectiveness of candidate DFT decorporation agents in vivo. Using europium as a model for americium, MRI studies determine the whole body distribution of europium after intratracheal or intravenous administration and serially follow the effects of oral administration of candidate DFT chelators on body distribution and elimination (Phase VII). Because of the hazards, analytic limitations and costs of working with americium, a surrogate metal is used. Europium has already been demonstrated to be an excellent model for americium in the development of decorporation agents [Gorden, et al., Rational design of sequestering agents for plutonium and other actinides, Chem Rev 2003; 103:4207-4282.] Eu(III) has been examined for use in MR contrast both as a paramagnetic agent in T2* studies [Fossheim, et al., Lanthanide-based susceptibility contrast agents: assessment of the magnetic properties, Magn Reson Med 1996; 35:201-206] and as a chemical exchange saturation transfer (CEST) agent with magnetization transfer techniques [Trokowski, et al., Cyclen-based phenylboronate ligands and their Eu3+ complexes for sensing glucose by MRI, Bioconjug Chem 2004; 15:1431-1440; Woessner, et al., Numerical solution of the Bloch equations provides insights into the optimum design of PARACEST agents for MRI, Magn Reson Med 2005; 53:790-799; Zhang, et al., A paramagnetic CEST agent for imaging glucose by MRI, J Am Chem Soc 2003; 125:15288-15289; Zhang, et al., A novel europium(III)-based MRI contrast agent, J Am Chem Soc 2001; 123:1517-1518.] Because lanthanide-based contrast agents are usually administered as chelates to avoid lanthanide toxicity, the optimal MR techniques for detection of Eu(III) administered in solution as the chloride (EuCl3) have not yet been established [Supkowski, et al., Displacement of Inner-Sphere Water Molecules from Eu(3+) Analogues of Gd(3+) MRI Contrast Agents by Carbonate and Phosphate Anions: Dissociation Constants from Luminescence Data in the Rapid-Exchange Limit, Inorg Chem 1999; 38:5616-5619.] A series of preliminary studies are carried out to optimize MR protocols for detection of Eu(III) and validate the results with measurement of Eu tissue concentrations using ICPMS as described above. MR studies are carried out at the Hatch Magnetic Resonance Research Center at Columbia University. High-resolution three-dimensional images of the rats are acquired using a Bruker AVANCE 400 whole body magnetic resonance system with a 9.4 T vertical-bore magnet, a MiniAHS/RFO mini-imaging in vivo probe, 0.75 G/cm/A actively shielded gradients, security box, and an animal handling system for exchangeable resonator/surface coil with the BioTrig system. Estimation of organ volumes and weights are carried out as previously described [Tang, et al., High-resolution magnetic resonance imaging tracks changes in organ and tissue mass in obese and aging rats, Am J Physiol Regul Integr Comp Physiol 2002; 282:R890-899] and estimation of Eu concentration is made on a per voxel basis. Rats are fasted overnight prior to imaging. EuCl3, 0.4 mL, dissolved in sterile 0.9% NaCl, are given by single intratracheal instillation or intravenous administration via the tail vein. At the end of study, rodents are sacrificed and estimates of organ concentrations verified by ICPMS. The desferrithiocin analogue chosen for this study will have completed stage VI of the proposed protocol, FIG. 4. At this point the europium clearing efficiency, dose response, organ distribution, pharmacokinetics and 30 day toxicity profile have been completed. In addition, the efficiency with which the ligand removes the metal from kidney, liver, lung, and bone have already been determined. The thirty day dosing schedule for the MRI assessment are predicated on this data. The study provides a dynamic picture of how the ligand mobilizes and clears the metal from various organ systems and the information compared with the other pharmacodynamic parameters

Decorporation of U(VI), Th(IV) and Eu(III) in Primates. The primate studies begin with Eu(III) (Phase VII). The protocol follows iron clearance studies in primates [Bergeron, et al., A Comparison of Iron Chelator Efficacy In Iron-Overloaded Beagle Dogs and Monkeys (Cebus apella), Comp Med 2004; 54:664-672] with some modifications. Five primates are given Eu(III) SC at a dose of 0.5 mg/kg. Three animals are given a chelator based on the results of the rodent studies with Eu(III). Two additional monkeys serve as Eu(III) controls. In the first experiment, the decorporation agent is administered PO at a dose of 300 μmol/kg 1 h post Eu(III). Urine and stool are collected for three days and assessed for their metal content. The animals are rested for 14 d and the experiment repeated; this time the chelator is not given until 2 h post metal exposure. This cycle is repeated next at 4 h post Eu(III) exposure. Fourteen days later, in a final experiment, the same 5 animals are given U(VI) and Th(IV) SC, each at a dose of 0.5 mg/kg. One hour post metal exposure, three of the monkeys are given a chelator PO at a dose of 300 μmol/kg. Two animals will serve as U(VI) and Th(IV) controls. Again, the choice of ligand are based on rodent studies; the ligand which decorporates both Th(IV) and U(VI) most effectively are selected. Urine and feces are collected for two days post drug. A longer collection time in this instance is not feasible, as primate metabolic cages must be cleaned every day. This would be too cumbersome in this experiment because of radiation safety issues. After this experiment, all five primates are euthanized and levels of U(VI), Th(IV) and Eu(III) are measured in kidney, liver, lung, and bone using ICPMS. We also measure chelator levels in these same tissues.

The invention provides DFT-based chelating agents for decorporation of radionuclides. Our past systematic structure-activity studies have allowed the design and synthesis of analogues and derivatives which retain the exceptional iron-chelating activity of DFT while eliminating the adverse effects. The hypothesis underlying the invention is that a similar approach can be adopted to utilize the DFT platform for the design of ligands that will effectively decorporate actinides. Building on past experience, the results of extensive studies of iron chelation in rodents and primates, and a wide-ranging review of the available scientific literature, the ligand basis set shown in Table 2 good available candidates at present for the decorporation of U(VI), Th(IV) [a surrogate for Pu(IV)] and Eu(III) [a surrogate for Am(III)]. As shown schematically in FIG. 4, systematic investigations in rodents (including dose-response, pharmacologic, toxicologic and histopathologic studies) identifies the DFT chelators in the ligand basis set that are most effective and least toxic for decorporation of U(VI), Th(IV) and Eu(III). We also examine an innovative new approach using MRI to characterize the action of candidate chelators of distribution and elimination of Eu(III) in rodents. Ultimately, the most promising candidate chelators are evaluated in a primate model to provide the best available evidence for efficacy in humans.

This focused approach has a very high probability of identifying DFT analogues that have substantial advantages over the currently available treatments (NaHC03 for U(VI); Ca- or Zn-DPTA for Th(IV) or Am(III) with respect to increased rates of actinide elimination, oral activity enhancing ease of mass casualty use, broader efficacy window (i.e., timing of product administration relative to radioactive contamination and duration of treatment). (S)-4′-(HO)-DADFT, 10, in Phase II clinical trials as an orally active chelator for the treatment of iron overload, significantly enhances U(VI) excretion in rodents (Table 1). Even more promising is (S)-4′-(CH3O)-DADMDFT, 11, which has successfully completed pharmacokinetic and toxicity studies in the NIH-RAID program, and produces significant and substantial reductions in renal U(VI), even when given orally 1 hour after U(VI) exposure. Accordingly, the invention identifies suitable lead DFT chelator(s) to enter further product development and provide critical data for the design of studies to prospectively assess efficacy in animals and safety in humans.

RATS: Male Sprague Dawley rats (200-250 g) are utilized for chelator organ/tissue distribution determinations. Additional male Sprague Dawley rats averaging 4-5 months of age (400 g) are utilized for the bile duct cannulation procedures and the collection of urine and feces samples during a multiple dosing regimen.

Slightly smaller (250-300 g) male rats of the same strain are used in the chronic (30 day) toxicity studies.

Organ/Tissue Distribution: Male Sprague Dawley rats (200-225 g) are given a single 300 μmol/kg dose of a chelator of interest orally or parenterally (SC). The animals (n=3/route/timepoint) are sacrificed via exposure to CO2 gas at t=0.5, 1, 2, 4, and 8 h post-drug. The animals' kidney, liver, lung and bone (femur) are removed and assessed for their chelator (parent plus metabolite) content.

Bile Duct Cannulation: After an overnight fast, the animals (n=5 per group) are anesthetized using ketamine/xylazine given IP at a dose of 40-80 mg/kg and 8-10 mg/kg, respectively. The bile duct is cannulated using 22-gauge polyethylene tubing. The incisions are closed with 2-0 gut (muscle) and surgical staples (skin). All of the rats to be used in this part of the protocol are provided with an analgesic: buprenorphine, 0.01-0.05 mg/kg SC every 8-12 h. The initial dose of analgesic are administered while the animals are still recovering from the general anesthetic. Once surgery is completed the rats are given a single dose of U(VI), Th(IV) or Eu(III). The metals are given SC at a dose equivalent to 2.8 mg/kg of U(VI). A decorporation agent are given PO or SC (300 μmol/kg) immediately thereafter or 1, 2 or 4 h post-metal. Bile samples are collected at 3 h intervals for 24 h. Urine samples are taken at 24 h. The animals are euthanized at the end of the experiment.

Rats: Urinary and Fecal Metal Clearance (Non-surgical): The initial screen of new compounds involves cannulating the rats' bile duct, administering a single dose of the test chelator and monitoring the urinary and biliary metal clearance for 24 hours. To further explore compounds that have been found to be effective actinide chelators, we would like to administer promising chelating compounds to metal-treated rats on a daily basis for four days. This allows determination of the cumulative urinary and fecal metal clearance induced by a given compound over a longer interval than is possible with a bile duct cannulated animal. The goal is to give a single SC injection of either U(VI), Th(IV) or Eu(III) and determine the urinary and fecal metal clearance of selected decorporation agents administered SC or orally by gavage once daily for four days. The initial dose of the chelators is given either immediately post metal, or not until 4 or 12 h post-metal exposure.

The rats are housed in individual metabolic cages and fasted overnight. The rats (n=5/metal/route) are given a single dose of U(VI), Eu(III), or Th(IV) SC. The metals are given at a dose equivalent to 2.8 mg/kg of U(VI). The animals are weighed daily and the chelators are given (1-2 μkg) SC or orally by gavage first thing in the morning and the animals are fed two hours post-drug. The rats have access to food for the remainder of the day and are again fasted overnight. This fasting is necessary because metal chelators that are administered orally will bind to any metals in the food that is in the gastrointestinal tract, thus masking or greatly decreasing the metal clearing efficiency of the compounds. Urine and feces samples are collected from the metabolic cages and are analyzed for metal content. The animals are not subjected to any surgical procedures or excessive restraint and are sacrificed at the end of the experiment. Tissues are then taken and assessed for their metal content.

Toxicity Studies: Male Sprague Dawley rats (250-300 g) are used in the chronic (30 d) toxicity trials. The rats (n=6 rats/dose level) receive the chelator at 1, 3 and 5 times the dose that causes the excretion of two times the metal excreted by the metal-only treated controls. The compound is given orally by gavage or SC once daily for 30 days. Control rats are given an equivalent volume of vehicle. Any animal showing signs of pain, distress, or discomfort due to the toxicity studies are sacrificed. A necropsy is performed whenever an animal is sacrificed, or at the conclusion of the experiment. Routine histopathology is carried out on selected tissues.

It is necessary to use the rats in order to establish the potential in vivo efficacy and toxicity of the metal chelators in question. The ability of the chelators to bind iron in vitro has already been established. However, the ability of a chelator to clear metals from an animal cannot be tested in vitro. Rodents have traditionally been used for the evaluation of metal clearance and provide a rapid and inexpensive primary screening of new chelators prior to their testing in the primates. Animal groupings are as described above. The number of rats needed varies according to the number of compounds evaluated per unit of time. Sample size calculation is consistent with what is in the literature and is identical to what we have used for years in the development of iron chelators.

The animals are housed in IACUC-inspected facilities and have access to veterinary care at all times. Animals used in the drug distribution/metabolism experiments receive a single dose of a decorporation agent orally or SC and pain and distress are minimal. The rodents used in the bile duct cannulation studies are provided with an analgesic: buprenorphine, 0.03-0.05 mg/kg SC every 8-12 h. The initial dose of analgesic is administered while the animals are still recovering from the general anesthetic.

Rats used in the multiple dosing regimen are not subjected to any surgical procedures or excessive restraint. Pain and distress are absent/minimal. Finally, animals used in the toxicity trials are weighed daily and are carefully monitored to their response to the drug (ruffled hair coat, staining around eyes and flares, activity level, etc). Animals showing signs of pain, distress, or weight loss ˜15% of their starting body weight are sacrificed via exposure to CO₂ gas.

At the end of the experiments, the rats are euthanized via exposure to CO₂ gas, followed by cervical dislocation and bilateral thoracotomy to ensure death. This is a safe and effective method of euthanasia and is consistent with the recommendations of the Panel on Euthanasia of the A VMA.

MRI Studies: In the studies, male Sprague Dawley rats, initially about 45 days of age and weighing 161 to 180 g are purchased from Charles River (SAS SO, Strain 400). The number of animals to be used is estimated by assuming that an average of about 16 animals are studied in each of the 18 months of the project, or a total of 288 animals. The overall goal of the studies in rats is the development of a new magnetic resonance imaging (MRI) method for evaluating the effectiveness of candidate DFT radionuclide decorporation agents in vivo. The radionuclide of interest is americium but because of the hazards, analytic limitations and costs of working with americium, a surrogate metal is used. Europium has been demonstrated to be an excellent model for americium in the development of decorporation agents[Gorden, et al., Rational design of sequestering agents for plutonium and other actinides, Chem Rev 2003; 103:4207-4282.] Using europium as a model for americium, MRI studies will determine the whole body distribution of europium after intratracheal or intravenous administration and serially follow the effects of oral administration of candidate DFT chelators on body distribution and elimination. Eu(III) has been examined for use in MR contrast both as a paramagnetic agent in T2* studies [Fossheim, et al., Lanthanide-based susceptibility contrast agents: assessment of the magnetic properties, Magn Reson Med 1996; 35:201-206] and as a chemical exchange saturation transfer (CEST) agent with magnetization transfer techniques.[Trokowski, et al., Cyclen-based phenylboronate ligands and their Eu3+ complexes for sensing glucose by MRI, Bioconjug Chem 2004; 15:1431-1440; Woessner, et al., Numerical solution of the Bloch equations provides insights into the optimum design of PARACEST agents for MRI, Magn Reson Med 2005; 53:790-799; Zhang, et al., A paramagnetic CEST agent for imaging glucose by MRI, J Am Chem Soc 2003; 125:15288-15289; Zhang, et al., A novel europium(III)-based MRI contrast agent, J Am Chem Soc 2001; 123:1517-1518.] Because lanthanide-based contrast agents are usually administered as chelates to avoid lanthanide toxicity, the optimal MR techniques for detection of Eu(III) administered in solution as the chloride (EuCl3) have not yet been established [Supkowski, et al., Displacement of Inner-Sphere Water Molecules from Eu(3+) Analogues of Gd(3+) MRI Contrast Agents by Carbonate and Phosphate Anions: Dissociation Constants from Luminescence Data in the Rapid-Exchange Limit, Inorg Chem 1999; 38:5616-5619] Initially, a series of preliminary studies are carried out to optimize MR protocols for detection of Eu(III) and validate the results with measurement of Eu tissue concentrations using ICPMS. MR studies are carried out at the Hatch Magnetic Resonance Research Center at Columbia University. After optimization and validation of the MR protocols, studies of the effects of the DFT chelators are carried out. The desferrithiocin analogue chosen for this study will have completed stage VI of the proposed research plan summarized in FIG. 4 above. At this point the europium clearing efficiency, dose response, organ distribution, pharmacokinetics and 30 day toxicity profile will have been completed. In addition, the efficiency with which the ligand removes the metal from liver, lung, and bone will have already been determined. The thirty day dosing schedule for the MRI assessment are predicated on this data. The study provides a dynamic picture of how the ligand mobilizes and clears the metal from various organ systems and the information compared with the other pharmacodynamic parameters.

In brief, high-resolution three-dimensional images of the rats are acquired using a Bruker AVANCE 400 whole body magnetic resonance system with a 9.4 T vertical-bore magnet, a MiniAHS/RFO.75 mini-imaging in vivo probe, 0.75 G/cm/A actively shielded gradients, security box, and an animal handling system for exchangeable resonator/surface coil with the BioTrig system. Estimation of organ volumes and weights are carried out as previously described [Tang, et al., High-resolution magnetic resonance imaging tracks changes in organ and tissue mass in obese and aging rats, Am J Physiol Regul Integr Comp Physiol 2002; 282:R890-899] and estimations of Eu concentration are made on per voxel basis. Experimentally, rats are fasted overnight prior to imaging. EuCb, 50 μmol/kg, dissolved in sterile 0.9% NaCl, pH 7.0, in a volume of 0.4 mL, are given by single intratracheal instillation or intravenous administration via the tail vein. Administration of the DFT analogues for orally active ligands are by gavage and for parenterally active agents by sc injection. With an experienced technician, rats accept gavage without distress or anesthesia.

No whole animal alternatives are available for these studies. Cell culture techniques would not provide the critical information needed about the systemic effects of the europium or of the DFT chelators or DFTEu(III) chelates. The extensive studies that precede the choice of the DFT analogue to be examined in the MRI studies will serve to minimize the number of animals that are examined with MRI.

The animals described in this study are housed in the facilities of the Institute of Comparative Medicine at Columbia University. The program for the care and use of laboratory animals at the Columbia University Medical Center is fully accredited by the American Association for the Accreditation of Laboratory Animal Care (AAALAC). The Laboratory Animal Resources provide space and housing for a wide variety of animal species used by the faculty. The Veterinary Medicine & Surgery Section ensures that all animals receive adequate veterinary care. To provide this, a comprehensive program is in place that includes the following components: quarantine; stabilization of newly arrived animals; infectious disease surveillance, treatment and control. The staff, in addition to veterinarians and supervisors, is composed of well trained and certified veterinary technicians. Animals in each room in the Laboratory for Animal Resources are observed daily for signs of illness by the animal technician responsible for providing husbandry. Medical records and documentation of experimental use are maintained on each animal's cage card. Routine veterinary medical care to all animals is provided by veterinary technicians under the direction of the attending veterinarian.

The only anticipated use of anesthesia are for the intratracheal and intravenous injections and for the MRI studies. The animals are briefly anesthetized with isoflurane gas (about 1.5 vol % at 1 L/min airflow) delivered via nose cone. After the intratracheal and intravenous injections or the MRI examinations are completed, the animals are allowed to recover. The rats are not expected to experience more than minimal pain or distress from the proposed studies. However, the animals are carefully monitored as discussed above, and any animals showing signs of pain, distress, or discomfort are sacrificed via exposure to CO₂ gas.

At the end of the experiment, the rodents are euthanized via exposure to CO₂ gas, followed by cervical dislocation and/or bilateral thoracotomy to ensure death. This is a safe and effective method of euthanasia and is consistent with the recommendations of the Panel on Euthanasia of the A VMA.

PRIMATES: Five male Cebus apella monkeys (2-4 kg) are utilized for the completion of the pharmacokinetics and metal clearance assessments. As the animals are jungle caught, their exact age is not known. The number of animals is consistent with what we have used in the development of our iron chelators.

Pharmacokinetics: Five animals are used for each kinetics experiment. The monkeys used in the kinetics study are fasted overnight. The following morning, the animals are sedated with Ketamine, 7-10 mg/kg 1 M, or Telazol, 0.03-0.05 mg/kg 1 M, given a single dose of atropine, 0.1 mg/kg 1 M, intubated and maintained on isoflurane gas, 1.5%. A t=0 blood sample are taken (2-3 mL) and the bladder are catheterized using a 5 French infant feeding tube.

The drug under investigation is administered either orally by gavage or parenterally (SC or IV) at a dose of 300 μmol/kg. Blood samples (2-3 mL) are drawn (times are approximate) at t=0, 0.5, 1, 2, 3, 4, 6 and 8 hours post-drug. Urine samples are taken via the catheter at t=0, 1, 2, 3 and 4 hours post-drug. The 14-21 mL of blood removed during the kinetics experiments is much less than the recommended maximum of 10 mL/kg. The monkeys are returned to their normal cages and are continuously observed until they are able to maintain themselves in a sitting position and are able to move about in their cages.

The animals are then resedated with Telazol at the 6 and 8 hour time points. The animals are fasted throughout the experimental period and are fed after the 8 hour time point. The monkeys are used in a pharmacokinetics study no more than once every 2 weeks.

Metal Clearance Studies: The same five male Cebus apella primates are used to assess the metal clearing efficiency of the new ligands. At the start of the Eu(III) clearance studies, the animals are sedated with Ketamine, 7-10 mg/kg 1 M, or Telazol 0.03-0.05 mg/kg 1M and bled for a baseline complete blood count (CBC) and blood chemistry. They are then transferred to the metal-free metabolic cages and started on a low-metal liquid diet. The animals are sedated on day “0” and given a single low (0.5 mg/kg) SC dose of Eu(III). The decorporation agent is given orally at a dose of 300 μmol/kg 1, 2 or 4 h post-metal. Urine and fecal collections continue from day −1 to day +3. At the end of each experiment, the animals are resedated and bled for post-drug blood analyses and transferred back to their normal cages. The animals are allowed a resting period of at least two weeks between studies. In a final experiment using U(VI) and Th(IV), the monkeys are sedated with Telazol. The primates are then given low (0.5 mg/kg) SC doses of both Th(IV) and U(VI). The test chelator is given PO 1 h later at a dose of 300 mol/kg. Urine and feces samples are collected for an additional 2 days. At the conclusion of this final assessment the animals are euthanized as described below and tissues taken for histology and determination of chelator/metal levels.

Rats, mice and other laboratory animals absorb and excrete iron and other metals in a manner that differs significantly from that of humans. To avoid doing costly human clinical trials with chelators that are ineffective, it is necessary to determine the efficacy in monkeys, whose iron metabolism closely resembles that of humans. The number of animals projected is necessary in order to provide statistically meaningful data and are consistent with what was used in the development of iron chelators.

The animals are housed in IACUC-inspected facilities and have access to veterinary care at all times. The monkeys are given periodic examinations by the veterinarians and are routinely monitored for fecal and blood-borne parasites, as well as tested for tuberculosis. In addition, a CBC and blood chemistry are performed before and after each study and the veterinary staff assesses any variation from the norm.

Prior to any procedure, the primates are sedated with Ketamine, 7-10 mg/kg 1 M, or Telazol, 0.03-0.05 mg/kg 1 M. During the metal clearing experiments the animals move freely in large metabolic cages and display no signs of stress, discomfort, or behavioral abnormalities. Once all of the projects described are completed, the monkeys are sedated with Ketamine, 7-10 mg/kg 1 M, or Telazol, 0.03-0.05 mg/kg 1M and then euthanized by the administration of sodium pentobarbital, 100 mg/kg IV. Extensive tissues are taken and evaluated for histopathology as well as for chelator/metal content. This is a safe and effective methods of euthanasia and is consistent with the recommendations of the Panel on Euthanasia of the AVMA.

Desferrithiocin Analogue-Induced Excretion of Uranium. In order to compare results with previous chelator-induced iron excretion data, the uranium clearance studies were carried out in a bile duct-cannulated rat model. The animals were given uranyl acetate dihydrate SC at a dose of 5 mg/kg (the actual dose of uranium is 2.8 mg/kg). The chelators were given IP, SC or PO at times relative to uranium exposure, indicated in Table 1. Bile and urine samples were collected for 24 hours after dosing. Kidneys were removed from selected animals and tissue uranium concentration measured. At least three animals were utilized in each experimental group. Data from two separate control studies (uranyl acetate/ no chelator) are combined for a total of 14 control animals. All uranium concentrations were measured using ICPMS. The data are reported as the total quantity of uranium excreted [urine +bile]; the mode of excretion [urine/bile] is also given. In addition the percentage of the administered dose of uranium cleared and chelator-induced uranium excretion vs the controls is also given. Four positive controls were evaluated: DTPA (1, Table 1) given as its trisodium calcium salt, DFO (2), N,N -bis(2-hydroxybenzyl)ethylenediamine-N,N-diacetic acid, monosodium salt (NaHBED, 3) and the hydroxypyridone CP94 (4) shown to bind uranium. The total metal cleared after 24 h in control rats was 10% of the administered dose. When DTPA, 1, was given IP or PO at 300 μmol/kg immediately after uranium, the excretion was 17% and 8% of the administered dose respectively (p>0.05). However, when the drug was given IP at a dose of 600 μmol/kg immediately post metal, the uranium excretion was significant, 20% (p<0.005). This is consistent with previously published data using a similar animal model.² DFO (2) and CP94 (4) given IP immediately post uranyl acetate were ineffective. NaHBED (3) marginally improved clearance (Table 1).

The scenario was very different with the DFT analogues. When (S)-4′-(CH3O)-DADFT (5) was given IP at 300 μmol/kg immediately after the uranium, metal excretion increased to 20% (p<0.003), but dropped to within error of control when given 0.5 h post metal. With (S)-4,5dihydro-5,5-dimethyl-2-(2-hydroxyphenyl)-4-thiazolecarboxylic acid [(S)-5,5-(CH₃)₂-DADMDFT, 6] given IP immediately post uranium, the excretion was 26% (p<0.002), but was again insignificant when given 0.5 h post metal. (S)-4′-(HO)-DADFT (7), the drug in clinic for iron overload, given IP immediately post metal, increased uranium excretion to 19% (p<0.05), while the corresponding polyether, (S)-4,5-dihydro-2-[2-hydroxy-4-(3,6,9-trioxadecyloxY)Phenyl]-4-methyl-4-thiazolecarboxylic acid [(S)-4′-(HO)-DADFT-PE (8)], given IP immediately post metal, raised uranium excretion to 22% (p<0.001, Table 1).

The most promising analogue in the (S)-2-(2,4-dihydroxyphenyl)-4,5-dihydro-4thiazolinecarboxylic series is the methoxy analogue (S)-4′-(CH3O)-DADMDFT (9). Given IP at 300 μmol/kg 0.5 h before uranyl acetate or immediately thereafter, nearly 30% of the metal was cleared (Table 1). This was also true if the drug was given IP 0.5 or 2 h post metal. The drug retains some efficacy given 4 h post uranium; 16% of the metal is excreted (p<0.006). When the chelator was given SC immediately post uranium, 40% of the administered metal was cleared. The increased excretion is probably associated with a slower absorption of the ligand. The data from rodents given the drug orally is even more encouraging (Table 1). When the chelator was administered PO at a dose of 300 μmol/kg immediately post metal, clearance was 23% (p<0.001), indicating very good oral bioavailability. At 2 h post metal the clearance was still significant, 18%, (p<0.005). When the dose of the chelator was increased to 600 μmol/kg and given PO 0.5 or 1 h post uranium, the clearance was 25% (p<0.001) and 26% (p<0.006) respectively (Table 1).

TABLE 1

| | | | | | Uranium | Chelator- | | % of |
|---|---|---|---|---|---|---|---|---|
| | | | | | Excretion | Induced U | | Administered |
| | | Dose | | Drug | (μg/kg) | Excretion/ | P vs | Dose |
| Compound/Structure | N | (μmol/kg) | Route | Timing | [urine/bite] | Control | Control | Cleared |
| Control Uranyl Acetate 5 mg/kg SC | 14 | — | — | — | 282 ± 124 | — | — | 10 |
| (Actual Uranium Dose = 2.8 mg/kg) | | | | | [100/0] | | | |
| 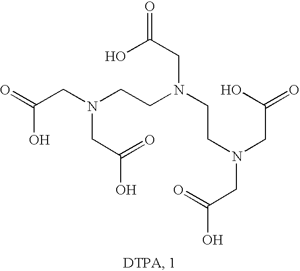 | 5 | 300 | IP | Immediate | 469 ± 271 [99/1] | 1.7 | NS | 17 |
| | 5 | 300 | PO | Immediate | 235 ± 205 | 0.8 | NS | 6 |
| | | | | | [100/0] | | | |
| | 5 | 600 | IP | Immediate | 546 ± 147 | 2.0 | <0.005 | 20 |
| | | | | | [100/0] | | | |
| 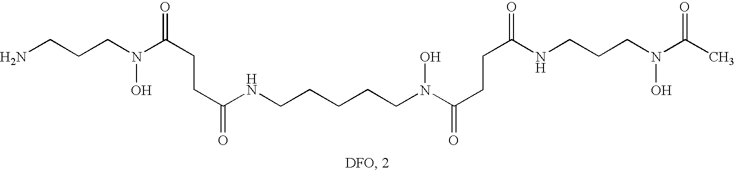 | 5 | 300 | IP | Immediate | 438 ± 191 [100/0] | 1.6 | NS | 16 |
| 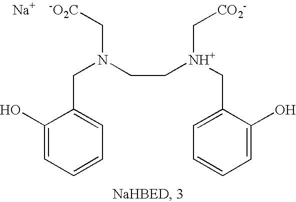 | 5 | 150 | IP | Immediate | 483 ± 119 [97/3] | 1.7 | <0.01 | 17 |
| 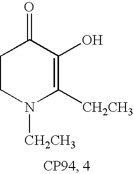 | 5 | 450 | IP | Immediate | 302 ± 171 [100/0] | 1.1 | NS | 11 |
| 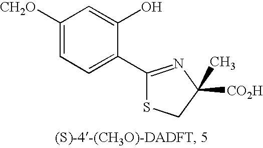 | 4 | 300 | IP | Immediate | 568 ± 133 [59/41] | 2.0 | <0.003 | 20 |
| | 4 | 300 | IP | 0.5 h Post | 413 ± 148 | 1.5 | NS | 15 |
| | | | | | [37/63] | | | |
| 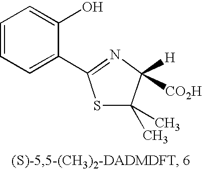 | 5 | 300 | IP | Immediate | 743 ± 189 [8/92] | 2.6 | <0.002 | 26 |
| | 4 | 300 | IP | 0.5 h Post | 200 ± 47 | 0.7 | NS | 7 |
| | | | | | [37/63] | | | |
| 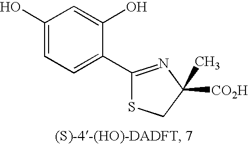 | 4 | 300 | IP | Immediate | 523 ± 180 [92/8] | 1.9 | <0.05 | 19 |
| 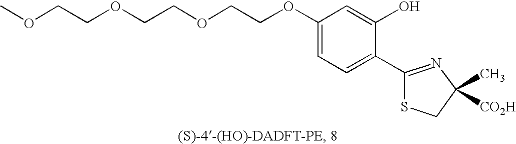 | 5 | 300 | IP | Immediate | 626 ± 123 [35/65] | 2.2 | <0.001 | 22 |
| 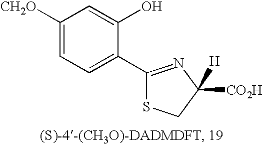 | 4 | 300 | IP | 0.5 h Pre | 815 ± 211 [48/52] | 2.9 | <0.006 | 29 |
| | 4 | 300 | IP | Immediate | 838 ± 166 | 3.0 | <0.002 | 30 |
| | | | | | [64/36] | | | |
| | 4 | 300 | SC | Immediate | 1130 ± 245 | 4.0 | <0.002 | 40 |
| | | | | | [13/87] | | | |
| | 5 | 300 | IP | 0.5 h Post | 917 ± 177 | 3.3 | <0.001 | 33 |
| | | | | | [16/84] | | | |
| | 5 | 300 | IP | 2 h Post | 847 ± 335 | 3.0 | <0.009 | 30 |
| | | | | | [46/54] | | | |
| | 7 | 300 | IP | 4 h Post | 444 ± 119 | 1.6 | <0.006 | 16 |
| | | | | | [66/34] | | | |
| | 5 | 300 | PO | Immediate | 642 ± 101 | 2.3 | <0.001 | 23 |
| | | | | | [43/57] | | | |
| | 8 | 300 | PO | 2 h Post | 507 ± 182 | 1.8 | <0.005 | 18 |
| | | | | | [53/47] | | | |
| | 6 | 600 | PO | 0.5 h Post | 698 ± 166 | 2.5 | <0.001 | 25 |
| | | | | | [24/76] | | | |
| | 3 | 600 | PO | 1 h Post | 723 ± 128 | 2.6 | <0.006 | 26 |
| | | | | | [28/72] | | | |
| Control: Uranyl Acetate 5 mg/kg SC | 14 | — | — | — | 282 ± 124 | — | — | 10 |
| (Actual Uranium Dose = 2.8 mg/kg) | | | | | [100/0] | | | |
| 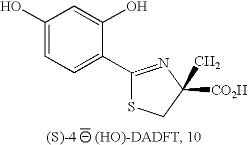 | 4 | 300 | IP | Immediate | 523 ± 180 [92/8] | 1.9 | <0.05 | 19 |
| 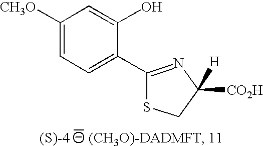 | 4 4 4 5 5 7 5 8 6 3 | 300 300 300 300 300 300 300 300 600 600 | IP IP SC IP IP IP PO PO PO PO | 0.5 h Pre Immediate Immediate 0.5 h Post 2 h Post 4 h Post Immediate 2 h Post 0.5 h Post 1 h Post | 815 ± 211 [48/52] 838 ± 166 [64/36] 1130 ± 246 [13/87] 917 ± 177 [16/84] 847 ± 335 [46/54] 444 ± 119 [66/34] 642 ± 101 [43/57] 507 ± 182 [53/47] 698 ± 166 [24/76] 723 ± 126 [28/72] | 2.9 3.0 4.0 3.3 3.0 1.6 2.3 1.8 2.5 2.6 | <0.006 <0.002 <0.002 <0.001 <0.009 <0.006 <0.001 <0.005 <0.001 <0.006 | 29 30 40 33 30 16 23 18 25 26 |
| 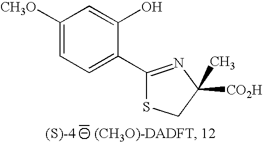 | 4 4 | 300 300 | IP IP | Immediate 0.5 h Post | 568 ± 133 [59/41] 413 ± 148 [37/63] | 2.0 1.5 | <0.003 NS | 20 15 |
| 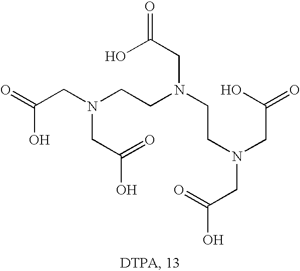 | 5 5 5 | 300 300 600 | IP PO IP | Immediate Immediate Immediate | 469 ± 271 [99/1] 235 ± 205 [100/0] 548 ± 147 [100/0] | 1.7 0.8 2.0 | NS NS <0.005 | 17 8 20 |
| 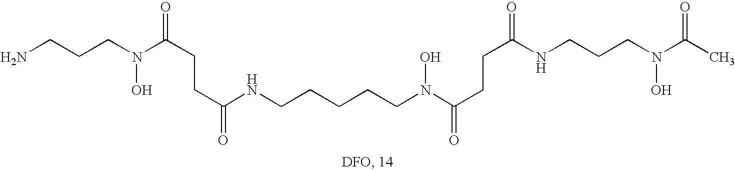 | 5 | 300 | IP | Immediate | 438 ± 191 [100/0] | 1.6 | NS | 16 |
| 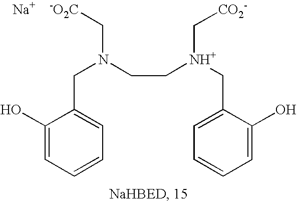 | 5 | 150 | IP | Immediate | 483 ± 119 [97/3] | 1.7 | <0.01 | 17 |
| 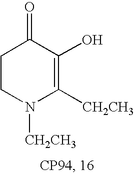 | 5 | 450 | IP | Immediate | 302 ± 171 [100/0] | 1.1 | NS | 11 |
| 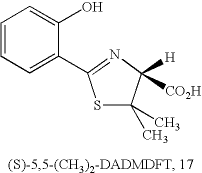 | 5 4 | 300 300 | IP IP | Immediate 0.5 h Post | 743 ± 189 [8/92] 200 ± 47 [37/63] | 2.6 0.7 | <0.002 NS | 26 7 |
| 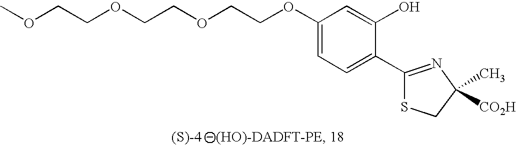 | 5 | 300 | IP | Immediate | 626 ± 123 [35/65] | 2.2 | <0.001 | 22 |

TABLE 2
Iron Clearing Activity of Proposed Desferrithiocin Analogues when administered
Orally to C. Apella Primates and the Partition Coefflcients of the Compounds

| Cysteine-Derived Compounds | | | α-Methylcysteine-Derived Compounds | | |
|---|---|---|---|---|---|
| Desferrithiocin | Iron Clearing | | Desferrithiocin | Iron Clearing | |
| Analogue | Efficiency | log_{b} | Analogue | Efficiency | log_{b} |
| (cpd. no.) | (%)^{a} | Pₐₚₚ | (cpd. no.) | (%)^{a} | Pₐₚₚ |
| 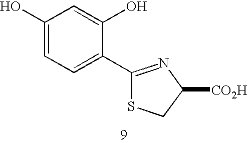 | 4.2 ± 1.4^{c} [70/30] | −1.33 | 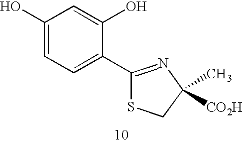 | 13.4 ± 5^{d} [86/14] | −1.05 |
| 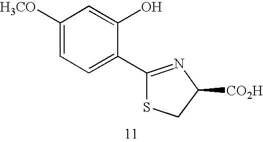 | 16.2 ± 3.2^{c} [81/19] | −0.89 | 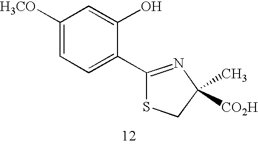 | 24.4 ± 10.8^{d} [91/9] | −0.70 |
| 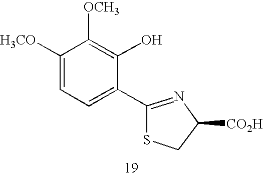 | 12.4 ± 6.2 [94/6] | −1.28 | 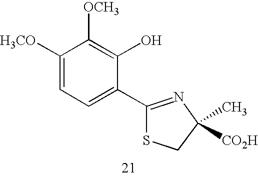 | | −0.95 |

_{^{a}}In the monkeys [n = 4 (9, 10, 11, 18), 6 (19), 7 (12)], the dose was 150 μmol/kg. The efficiency of each compound was calculated by averaging the iron output for 4 days before the administration of the drug, subtracting these numbers from the 2-day iron clearance after the adminstration of the drug, and then dividing by the theoretical output; the result is expressed as a percent. The relative percentages of the iron excreted in the stool and urine are in brackets. ^{b}Data are expressed as the log of the fraction in the octanol layer (log Pₐₚₚ); measurements were done in TRIS buffer, pH 7.4, using a ‘shake flask’ direct method. The values obtained for compounds 9-12 are from ref 36. ^{c}Data are from ref 19. ^{d}Data are from ref 17. ^{a}Data are from ref 36. ^{f}Data are from ref 38.

Reference is made to Intl. Pub. No. WO/2006107626 Al and U.S. Ser. No. 60/966,539, entitled “Desferrithiocin Polyether Analogues”, inventor Raymond J. Bergeron, Jr., filed Mar. 15, 2007 (our Ref. T2315-11335PV01), the entire contents and disclosures of which are incorporated herein by reference.”

1. A pharmaceutical composition comprising a non-toxic effective amount of an actinide decorporation agent and a pharmaceutically acceptable carrier therefore, said actinide decorporation agent comprising a hexacoordinate desferrithiocin analog capable of chelating an actinide in vivo.

2. A method for removing an actinide from the tissue of a human or nonhuman mammal comprising administering to said mammal a non-toxic effective amount of the actinide decorporation agent of claim 1 to chelate or form a complex with said actinide and removing said chelate or complex by excretion.

3. An article of manufacture comprising a container housing an actinide decorporation agent of claim 1 and a package insert or indicia on said container that indicates that said agent is useful for the removal of actinides from human and non-human mammals.
